(19) United States
(12) Patent Application Publication
(10) Pub. No.: US 2018/0325765 A1
(43) Pub. Date: Nov. 15, 2018

(54) LOCOMOTION ASSISTANCE MEANS

(71) Applicant: Guy WILMINGTON, Manuka (AU)

(72) Inventor: Guy WILMINGTON, Manuka (AU)

(21) Appl. No.: 15/774,834

(22) PCT Filed: Nov. 3, 2016
(86) PCT No.: PCT/AU2016/051042

§ 371 Date: May 9, 2018

(30) Foreign Application Priority Data
Nov. 9, 2015 (AU) 2015904580

(51) Int. Cl. A61H 3/00; A61B 5/11; A61H 1/02; A41D 1/08

(52) U.S. Cl. CPC A61H 3/00; A61H 2201/5069; A61H 1/0262; A61H 1/0266; A41D 1/08; A61B 2505/09; A61H 2201/1207; A61H 2201/163; A61H 2003/007; A61H 2201/1642; A61H 2201/165; A41D 2400/32; A61H 2201/5007; A61H 2201/5038; A61B 5/1122

(57) ABSTRACT

An appliance for assisting locomotion of a subject, including a paired ankle joint controller configured to return an ankle joint of the subject to a neutral position; a paired electromechanical knee joint controller configured to move each knee joint of the subject through a range of motion required for locomotion; a paired electromechanical hip joint controller configured move each hip joint of the subject through a range of motion required for locomotion; an electronic appliance control unit in electrical or data communication with (i) the paired electromechanical knee joint controller and (ii) the paired electromechanical hip joint controller. The electronic control unit configured to coordinate the movement of each knee joint and each hip joint of the subject so as to cause or assist locomotion of the subject. Also provided are methods for programming the electronic control unit.

### RELATED APPLICATIONS

The present application claims priority from Australian Provisional Patent Application No. 2015904580, having the filing date of 9 Nov. 2015, the specification as filed being incorporated herein by reference in its entirety.

### FIELD OF THE INVENTION

The present invention is in the field of mobility aids, and particularly the field of electromechanical aids that assist an individual to walk.

### BACKGROUND TO THE INVENTION

Mobility aids for paraplegics and the frail have not advanced significantly since 1655 with the development of the self-propelled wheel chair. While there have been some recent advances in the development of exoskeleton-type appliances that attach to a subject's legs, such appliances have had limited success in providing independent bipedal locomotion in an acceptable manner.

Current approaches which rely on the existence of exoskeletons are deigned to partially or fully bear the weight of the subject. Moreover, the exoskeleton act as an attachment point which mobility actuators (such as electric motors and hydraulic rams) are attached. As a consequence, in order to achieve the necessary performance these appliances are bulky, require a sizeable power supply and are generally unable to operate without a need for the subject bear some of their own body weight through the use of crutches or the like.

A further problem is that prior art appliances can be difficult for the subject to control. Prior art appliances typically require complex control systems to first sense and then control the amount of movement required to apply the precise force needed to achieve the desired outcome. Some attempts of the prior art require complex input from the subject so as to activate the relevant components of the appliance and any motors involved. Even where some reasonable level of control is mastered by a subject, only an abnormal gait may be achievable.

Yet a further problem is that prior art appliances (due to their bulkiness) must be worn exterior to the clothing, and therefore provide a highly visible sign that the subject has a mobility problem. Many persons of impaired mobility are aware of the social stigma of being considered “disabled” and therefore find such appliances to be generally undesirable.

It is an aspect of the present invention to provide improved means for assisting the locomotion of a mobility impaired person. The improvement may be in any one or more of weight, simplicity of construction, simplicity of use, quality of gait achieved, economics, aesthetics, subject acceptability, or indeed any other advantage. In another aspect the present invention provides an alternative to prior art means for assisting locomotion in mobility impaired persons.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each provisional claim of this application.

### SUMMARY OF THE INVENTION

In a first aspect, but not necessarily the broadest aspect the present invention provides an appliance for assisting locomotion of a subject in need thereof, the appliance comprising paired ankle joint control means configured (either alone or in combination with other component(s)) to return the ankle joint to a neutral position during locomotion.

In one embodiment of the first aspect, the neutral position is where the long axis of the lower leg is about 90 degrees to a plane described by the sole of the foot.

In one embodiment of the first aspect, each of the paired ankle joint control means returns to the ankle joint to a neutral position by way of a biasing means.

In one embodiment of the first aspect, the biasing means is a torsion spring.

In one embodiment of the first aspect, the appliance comprises paired electromechanical knee joint control means configured (either alone or in combination with other component(s)) to move each knee joint through a range of motion required for locomotion,

In one embodiment of the first aspect, each of the paired electromechanical knee joint control means comprising biasing means configured to substantially counteract some or all of the gravitational force occasioned on the subject when moving from a seated position to a standing position, so as to extend the knee joint and allow the subject to assume a standing position.

In one embodiment of the first aspect, the biasing means is a leaf spring or a torsion spring.

In one embodiment of the first aspect, each of the paired electromechanical knee joint control means comprises at least one electric motor configured to (either alone or in combination with other component(s)) effect the range of motion of the knee joint required for locomotion.

In one embodiment of the first aspect, each of the paired electromechanical knee joint control means comprises one or more of a gear, a drive, a pulley; each of which is in direct or indirect operable connection with the at least one electric motor; the one or more of a gear, a drive, a pulley being configured to (either alone or in combination with other component(s)) effect the range of motion of the knee joint required for locomotion.

In one embodiment of the first aspect, the appliance comprises paired electromechanical hip joint control means configured (either alone or in combination with other component(s)) to move each hip joint through a range of motion required for locomotion,

In one embodiment of the first aspect, each of the paired electromechanical hip joint control means comprises at least one electric motor configured to (either alone or in combination with other component(s)) effect the range of motion of the hip joint required for locomotion.

In one embodiment of the first aspect, each of the paired electromechanical hip joint control means comprises one or more of a gear, a drive, a pulley; each of which is in direct or indirect operable connection with the at least one electric motor; the one or more of a gear, a drive, a pulley being configured to (either alone or in combination with other component(s)) effect the range of motion of the knee joint required for locomotion.

In one embodiment of the first aspect, each of the paired electromechanical hip joint control means is configured to rotate the femur forwards and/or backwards and/or outwards and/or inwards and/or effect axial rotation of the femur.

In one embodiment of the first aspect, the appliance comprises electronic appliance control means in electrical or data communication with (i) the paired electromechanical knee joint control means (where present) and/or (ii) the paired electromechanical hip joint control means (where present), the electronic appliance control means configured to coordinate the movement of each knee joint and/or each hip joint of the subject so as to cause or assist locomotion of the subject.

In one embodiment of the first aspect, the appliance comprises knee joint sensing means configured to sense the degree of bending or straightening of the knee, the sensing means being in electrical or data communication with the electronic appliance control means.

In one embodiment of the first aspect, the appliance comprises hip joint sensing means configured to sense the degree of movement and/or direction of movement and/or rotation of the femur relative to the pelvis, the sensing means being in electrical or data communication with the electronic appliance control means.

In one embodiment of the first aspect, the paired electromechanical hip joint control means (where present) and/or the paired knee joint control means (where present) is/are configured to operate in a coordinated manner via the electronic appliance control means so as to move the pelvis forwards and/or backwards and/or outwards and/or inwards and/or effect axial rotation of the pelvis.

In one embodiment of the first aspect, the electronic appliance control means accepts electrical signals and/or data from the knee joint sensing means (where present) and/or the hip joint sensing means (where present), and is configured to process the electrical signals and/or data so as to provide output electrical signals and/or data, the output electrical signals and/or data being communicated to any of the more or more motors of each of the paired electromechanical knee joint control means (where present) and/or each of the paired electromechanical hip joint control means (where present) so as to cause or assist for locomotion of the subject.

In a second aspect the present invention provides a method of programming an electronic control unit of an electromechanical appliance configured to cause or assist in the locomotion of a subject, the method comprising the steps of: providing a locomotion-capable subject, providing paired knee joint sensing means configured to sense the degree of bending or straightening of a knee, providing paired hip joint sensing means configured to sense the degree of movement and/or direction of movement and/or rotation of a femur relative to a pelvis, attaching sensing means to each knee joint and hip joint of the locomotion-capable subject, allowing the locomotion-capable subject to move from a seated position to a standing position and/or walk and/or jog and/or run and/or move from a standing position to seated position, recording over a period of time the electrical signals and/or data output by the paired knee joint sensing means and the paired hip joint sensing means so as to identify the normal range of motion of each of the knee joint and the hip joint of the locomotion-capable subject during any of moving from a seated position to a standing position and/or walking and/or jogging and/or running and/or moving from a standing position to seated position, and programming an electronic control unit of an electromechanical appliance with the recorded data, or data derived from the recorded data, such that an electromechanical device operably connected to the control unit and fitted to a locomotion-compromised subject can substantially replicate or approximate in the locomotion-compromised subject the normal range of motion of each of the knee joint and the hip joint of the locomotion-capable subject during any of moving from a seated position to a standing position and/or walking and/or jogging and/or running and/or moving from a standing position to seated position.

In one embodiment of the second aspect, the locomotion-capable subject substantially shares any one or more of the following parameters with the locomotion incapable subject for which the electronic control means is being programmed: weight, height, age, femur length, pelvis width, pelvis shape, tibia length, fibula length, foot length, foot width, hip joint mobility, knee joint mobility, ankle joint mobility, muscle mass of any muscle of the legs or hips.

In a third aspect there is provided computer readable medium having stored thereon data recorded by the method of the second aspect, or data derived from the data recorded by the method of the second aspect.

In one embodiment of the first aspect, the electronic appliance control means comprises the computer readable medium of the third aspect.

In one embodiment of the first aspect, the control means further comprises processor-executable software configured to accept as input the data stored on the computer-readable medium, and control the appliance so as to effect a required range of motion in the paired ankle joint control means and/or the paired electromechanical knee joint control means.

### BRIEF DESCRIPTION OF THE DRAWINGS

FIG. 1 shows a highly preferred appliance of the present invention as fitted to a subject.

FIG. 2 shows a spring-loaded hinged ankle brace, being a component of the highly preferred appliance shown in FIG. 1. 2A shows the brace in assembled form, and 2B in exploded form.

FIG. 3 shows an electromechanical knee brace sleeve, being a component of the highly preferred appliance shown in FIG. 1. 3A shows the brace in assembled form, and 3B in exploded form.

FIG. 4 shows the electromechanical hip brace, being a component of the highly preferred appliance shown in FIG. 1.

FIG. 5 shows a control unit being actuatable by a subject fitted with the highly preferred appliance shown in FIG. 1.

### DETAILED DESCRIPTION OF THE INVENTION INCLUDING PREFERRED EMBODIMENTS

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as are apparent to one of ordinary skill in the art from this disclosure, in one or more embodiments.

Similarly it should be appreciated that the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and from different embodiments, as are understood by those in the art.

In the claims below and the description herein, any one of the terms “comprising”, “comprised of” or “which comprises” is an open term that means including at least the elements/features that follow, but not excluding others. Thus, the term comprising, when used in the claims, should not be interpreted as being limitative to the means or elements or steps listed thereafter. For example, the scope of the expression a method comprising step A and step B should not be limited to methods consisting only of methods A and B. Any one of the terms “including” or “which includes” or “that includes” as used herein is also an open term that also means including at least the elements/features that follow the term, but not excluding others. Thus, “including” is synonymous with and means “comprising”.

The following description pertains to a highly preferred embodiment of the present invention, and is not limiting in any way on the scope of this application.

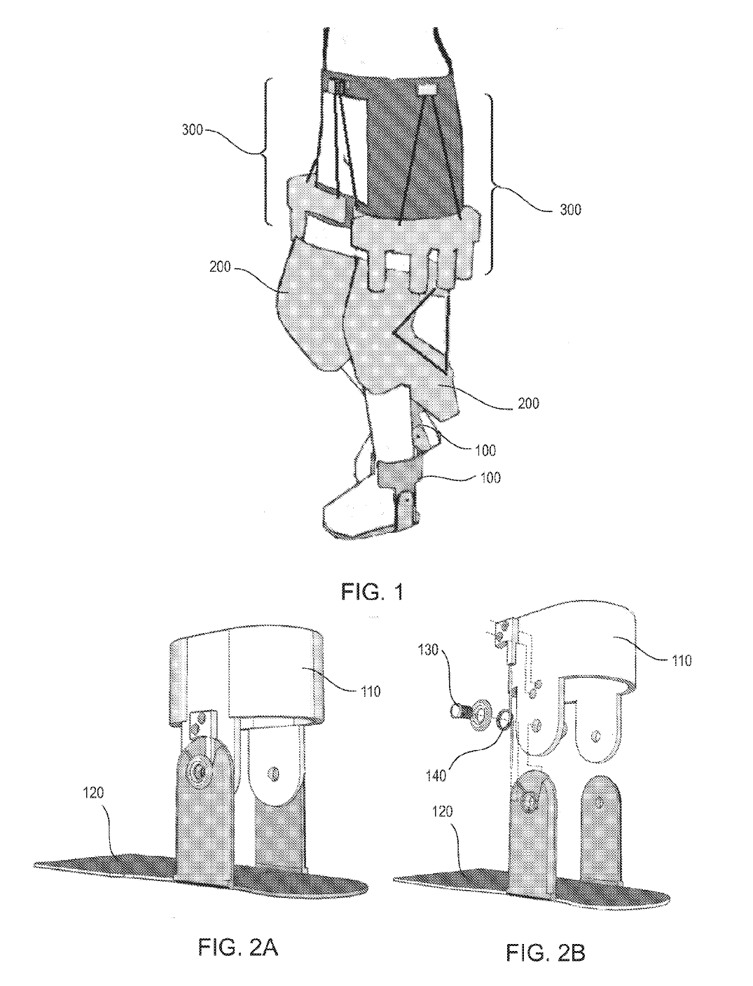

Reference is made to FIG. 1, showing an appliance of the present invention comprising paired ankle joint control means 100 (one of the pair in a preferred form being the spring loaded ankle brace described infra), paired electromechanical knee joint control means 200 (one of the pair in a preferred form being the electromechanical knee brace sleeve described infra), and paired electromechanical hip joint control means 300 (the pair being comprised in a preferred form by the electromechanical hip brace described infra).

### Spring Loaded Ankle Brace

Reference is made to FIG. 2, the spring loaded ankle brace comprising of: (a) an upper portion 110 secured to the lower end of the lower leg by means of a strap or similar device and secured by a buckle, hook-and-loop system or similar device, (b) a lower portion 120 in the form of a stirrup or similar shape that sits under the foot and is attached to the upper portion at either side of the ankle by means of; (c) rivets 130, or similar that enable the upper and lower portions to rotate with respect to one another at the ankle joint; and (d) two torsion springs 140 (one marked, and on both sides of the ankle, with ends attached to the upper and lower portions and pivoting at the ankle joint.

The spring loaded ankle brace may also include padding to reduce or eliminate the possibility of lesions or other injuries to the lower leg, ankle or foot due to repetitive use of the spring loaded ankle brace over long periods of time.

The geometry and shape of the upper and lower portions, the means of securing the upper portion to the lower end of the lower leg or the means of securing the upper and lower potions to each other can be altered to suit a particular subject. In this preferred embodiment, these components of the spring loaded hinged ankle brace act so as to form a secure yet comfortable means to: (i) associate the spring to the ankle joint; and (ii) enable the spring the exert force on the ankle joint such that when a person is standing on both feet the force produced by the spring is such that the ankle joint is held at right angles.

As to the springs (d), these are torsion springs or similar that are designed and situated such that they exert no force when the ankle is at right angles, but exert increasing force as the angle between the line of the foot and the lower leg decreases.

The amount of force exerted by each spring is expressed as:

(cosine (θ)×½ height of subject×weight of subject×gravitational force)/4

where θ is the angle between the line of the foot and the lower leg.

The purpose of the springs is two-fold: (1) to prevent the subject from falling forward when standing on two feet; and (2) to store and release force as the subject walks forward.

The spring-loaded ankle brace may be a stand-alone appliance, or may be incorporated into a shoe or a boot.

### Electromechanical Knee Brace Sleeve

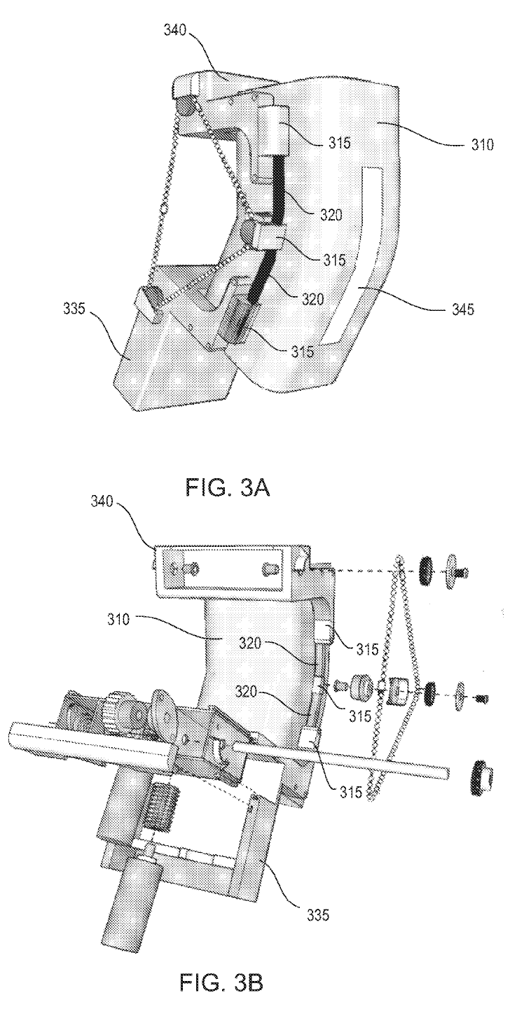

Reference is made to FIG. 3 showing an electromechanical knee brace sleeve comprised of: (a) a sleeve 310 made of flexible and elastic material that the subject pulls on over their knee joint leaving a portion above and below the knee joint; (b) webbing belt or similar material (not shown) secured by buckles, hook and loop tape or similar, above and below the knee joint to secure the sleeve above and below the knee joint to prevent it slipping; (c) anchor points 315 on the sleeve at both sides of the knee. These anchor points serve as the point of attachment for the springs 320 (d), lower electromechanical portion 325 (e), upper electromechanical portion 330 (f) and the straightening pulleys (g); (d) springs situated either side of the knee joint and attached to the flexible sleeve in a manner to exert force to straighten the knee joint; (e) a lower electromechanical portion 335 attached to the flexible sleeve (a) at anchor points (c) in which is housed and attached the small electrical motors, gears and pulleys used to bend and straighten the knee brace sleeve; (f) an upper electromechanical portion 340 attached to the flexible sleeve (a) at anchor points (c) used to bend and straighten the knee brace sleeve; (g) pulleys attached to the central anchor point across which the cables traverse, serving as a pivot point for the straightening of the knee brace sleeve; (h) a polymer based sensor 345 situated across the patella to measure the changes in the distance between the upper and lower electromechanical portions across the patella as the knee is bent and straightened; and (j) control lines (not shown) extending from the electromechanical knee brace sleeve to the electromechanical hip brace to convey sensory information from the flexible polymer based sensor (h) to the control unit; and command information from the control unit to the electric motors attached to the lower electromechanical portion (e).

The shape and materials of the knee brace sleeve (a), the location, shape and materials of the webbing belt or similar (b) and the location, shape and materials of the anchor points (c) can be used to suit a particular subject. The key features of these components is that, in combination, they provide sturdy points to which are attached the springs (d), the lower electromechanical portion (e), the upper electromechanical portion (f) and the straightening pulleys (g).

The springs (d) can be leaf springs, torsion springs or similar and are designed and installed that they offset the gravitational force induced torque at the knee joint generated when a subject sits and provide sufficient force, should the electromechanical portions not be contributing any force, to cause the subject to stand.

Given that there are four sets of springs, one each side of each knee, the amount of force exerted by each spring is expressed as:

(sine (θ)×length of subject's femur×weight of subject×(gravitational force+0.2))/4

where θ is the angle between the line of the upper and lower leg; and
0.2 is the inbuilt allowance of force to straighten the knee joint

By this arrangement the electromechanical portions are mainly operating to bend the leg against the inbuilt allowance of force to straighten the knee joint rather than having electromechanical devices of sufficient power to straighten a knee joint against gravitational force. In doing so, the force needed to be produced by the electromechanical portions is significantly reduced, thereby reducing the size, weight and power consumption of the same.

The lower electromechanical portion is comprised of: (i) a housing or framework which is attached to the flexible knee brace sleeve (a) at the two lower anchor points (c); (ii) multiple small electrical motors working in parallel and arranged vertically behind the knee joint approximate the calf muscle; (iii) worm gears to convert the rotation produced the electric motors, which is around a vertical axis to rotation around a horizontal axis parallel to the axis of the knee joint; and to prevent the force of the springs turning the electric motors in a reverse direction to that intended or requiring the electric motors to be always operational to maintain a desired bend in the knee joint (iv) a gear housing to encapsulate the lower portion pulleys. These pulleys are driven by the worm gears and, together with those in the upper electromechanical portion, work with a rope, notched rope or similar, to reduce the distance between the upper electromechanical portion and the lower mechanical portion thereby causing the knee to bend.

The upper electromechanical portion is comprised of: (v) a housing or framework which is attached to the flexible knee brace sleeve at the two lower anchor points In operation, the motors turn such that, in combination with the worm gears and pulley system they bend and straighten the knee in accordance with commands issued by the control unit (as shown in FIG. 5).

A variation of the embodiment described above and shown in FIG. 3, does not have a pulley in the upper portion of the knee brace sleeve or at the point of the joint itself. Rather the notched rope (or similar) terminates at, or is anchored to, the upper potion in two locations.

### Electromechanical Hip Brace

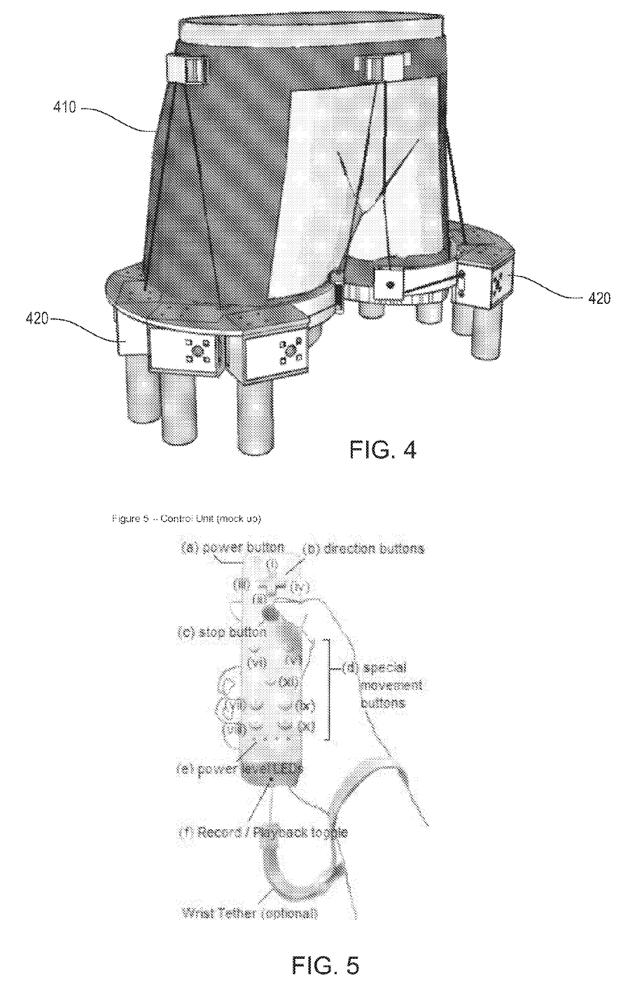

Reference is made to FIG. 4 showing an electromechanical hip brace comprised of: (a) a pair of shorts 410 made of flexible and elastic material that the subject pulls on and that covers the upper thigh of both legs and a portion of the pelvic area. Note that the groin and anal areas are not covered so that the subject can perform necessary bodily functions without the need to remove the electromechanical hip brace; (b) four (4) anchor points located equal distance around the waist area to which the electric motors are connected via notched cord or chain such that when the electric motors are activated the distance between the lower portions and the waist area is either reduced or lengthened; (c) two lower portions 420, one attached to each thigh area, that incorporate a minimum of four motors to give the necessary actuation via notched rope/chain or equivalent to move the hip joint; and (d) polymer based sensors (not shown) arranged to measure the movement of the hip in the forwards and backwards swing of the same during walking, standing, sitting etc. sideways swing of the same when the subject performs a side-step; and twisting of the leg about the axis of the femur as the subject rotates their body position.

The exact shape and materials of the flexible and elastic pair of shorts (a) is not germane and can be altered to suit an individual. The pertinent features are that: the shorts can be pulled on easily and do not ride down under normal use; and the groin and anal areas are not covered so that the subject can perform necessary bodily functions without removing the shorts.

The upper portion (b) is permanently affixed to the waist region of the flexible shorts (a) at a number of locations around the circumference and is secured, when worn, by a buckle or similar device. When the buckle is not secured there should be sufficient flexibility between the points where the upper portion is affixed to enable the subject to pull the electromechanical hip brace up and over their hips before closing the buckle and securing the electromechanical hip brace in position.

The lower portion (c) also provides the mounts for the: (i) front inner; (ii) front outer; (iii) back inner; (iv) back outer; as well as the point of connection for one end of the (vi) forward motion sensors, the (vii) sidewards motion sensors; (viii) the twisting motion sensors and the elastic rope (d).

Finally, the lower portion (c) is connected to the power source by means of flexible electrical wires and to the control unit (FIG. 5) by means of flexible electrical wires or other means of communication. Depending on the designed range, power requirements and power source performance the power source can be: attached directly to the upper portion of the electromechanical hip brace; carried in a back pack; or pulled behind in a tethered wheeled suitcase or the like.

The upper portion may also provide anchor points for braces or similar suspenders (f) that extend from the front of the upper portion over the subject's shoulders to be connected once again to the upper portion at the back. This optional addition may be required when the subject's anatomy is not suitably shaped such that the weight of the apparatus cannot be supported by the hips alone.

The lower portions (one for each leg) are permanently affixed to the thigh region of the flexible shorts (a) at a number of locations around the circumference thereof and are secured, when worn, using a webbing belt (or similar) with a buckle (or similar) to ensure that the lower portions do not rise up during use of the electromechanical hip brace.

The lower portions provide the mounts for the reference points for the: (i) front inner; (ii) front outer; (iii) back inner; and (iv) back outer motors.

In operation, the motors turn such that, in combination with the worm gears and pulley system would bend the hip joint: forward by tightening the pulley ropes attached to the front inner and front outer motors and relaxing the pulley ropes attached to the back inner and back outer motors; backwards by tightening the pulley ropes attached to the back inner and back outer motors and relaxing the pulley ropes attached to the front inner and front outer motors; outwards by tightening the pulley ropes attached to the front outer and back outer motors and relaxing the pulley ropes attached to the front inner and back inner motors; inwards by tightening the pulley ropes attached to the front inner and back inner motors and relaxing the pulley ropes attached to the back inner and back outer motors; and twisting by tightening the pulley ropes attached to the diagonally opposed motors; in accordance with commands issued by the control unit [FIG. 5].

### Control Unit

Reference is made to the control unit shown in FIG. 5 connected to the electromechanical hip brace (FIG. 4) by flexible electrical conductors/wires, WiFi or Bluetooth™ communications or similar and thence also to the electromechanical knee brace for the purpose of: collecting sensory data from the polymer based sensors positioned around the hip and at the knee; and issuing commands to the various electrical motors.

The control unit operates in the following two modes: Record. In this mode the whole apparatus is worn by an able bodied person of similar physical dimensions (leg length, overall height, weight, hip width etc) to that of the intended users and the sensor readings are recorded on an internal EPROM (or similar) and associated to a predefined movement profile; and Playback. In this mode the whole apparatus is worn by the intended subject.

When the subject selects a movement profile by clicking the corresponding button on the control unit, commands are sent by the control unit to the electromechanical knee brace and the electromechanical hip brace to ensure that the actual sensory data matches the pre-recorded sensory data for that point in time in the selected movement profile. For example, if the pre-recorded movement profile has at time point x a knee bend angle of y AND the actual knee bend sensor has a different sensory reading, then commands will be sent to the electrical motors on the lower portion of the electromechanical knee brace to bend the knee so that the actual sensory data matches the pre-recorded sensory data for time point x.

The control unit has the following: (a) power button to turn it and the apparatus on; (b) a set of directional buttons to control the direction of the walking gait; specifically: (i) forward; (ii) backward; (iii) veer left; (iv) veer right; (c) a large stop button. Pressing this button will stop all forward movement and return the apparatus/subject to an upright position; (d) special movement command buttons such as: (v) side-step right; (vi) side-step left; (vii) stand; (viii) sit; (ix) stair climb; (x) stair descend; and (xi) rotate; (e) a set of LEDs or similar that indicate the remaining power level in the power supply; and (f) a hidden toggle button or similar activated using a pin or similar to change the control unit between the Record and Playback modes.

The control unit may include an optional wrist tether to ensure that it is not dropped or become lost.

Alternatively, the functions of the control unit may be replicated by an application (App) on a ‘SmartPhone’, ‘Tablet’ or similar.

As will be apparent from the above, the present invention may involve the use of processors, (including microprocessors), software executable on such devices and the like to control one or more motors and/or to received sensor data and/or to process sensed data. Some methods may be computer executable. Moreover, systems may be required interconnect components such as processors, motors, sensors and the like.

Application software useful in the context of the present invention may be executable on any past, present or future operating system of a mobile communication device including Android™, iOS™, Windows™, Linux™ and the like.

The methods and systems described herein may be deployed in part or in whole through one or more processors that execute computer software, program codes, and/or instructions on a processor. The processor may be part of a server, client, network infrastructure, mobile computing platform, stationary computing platform, or other computing platform. A processor may be any kind of computational or processing device capable of executing program instructions, codes, binary instructions and the like. The processor may be or may include a signal processor, digital processor, embedded processor, microprocessor or any variant such as a coprocessor (math co-processor, graphic co-processor, communication co-processor and the like) and the like that may directly or indirectly facilitate execution of program code or program instructions stored thereon. In addition, the processor may enable execution of multiple programs, threads, and codes.

The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere.

Any processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through one or more computers that execute software. The computer may include one or more of memories, processors, computer readable media, storage media, ports (physical and virtual), communication devices, and interfaces capable of accessing other computers, and devices through a wired or a wireless medium, and the like. The methods, programs or codes as described herein and elsewhere may be executed by the computer. In addition, other devices required for execution of methods as described herein.

Methods, programs codes, calculations, algorithms and instructions useful in relation to the present invention may be implemented on or through mobile devices. The mobile devices may include navigation devices, cell phones, mobile phones, smart phones, mobile personal digital assistants, laptops, palmtops, netbooks, tablets and the like. These devices may include, apart from other components, a storage medium such as a flash memory, buffer, RAM, ROM and one or more computing devices. The computing devices associated with mobile devices may be enabled to execute program codes, methods, and instructions stored thereon.

Alternatively, the mobile devices may be configured to execute instructions in collaboration with other devices, such as a dedicated controller of the appliance. The mobile devices may communicate with the controller and configured to execute program codes.

The computer software, program codes, and/or instructions may be stored and/or accessed on computer readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks. Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The methods and/or systems useful in the present invention may be realized in hardware, software or any combination of hardware and software suitable for a particular application. The hardware may include a general purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a computer readable medium.

The Application software may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, a method of the present invention may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

The invention may be embodied in program instruction set executable on one or more computers. Such instructions sets may include any one or more of the following instruction types:

Data handling and memory operations, which may include an instruction to set a register to a fixed constant value, or copy data from a memory location to a register, or vice-versa, to store the contents of a register, result of a computation, or to retrieve stored data to perform a computation on it later, or to read and write data from hardware devices.

Arithmetic and logic operations, which may include an instruction to add, subtract, multiply, or divide the values of two registers, placing the result in a register, possibly setting one or more condition codes in a status register, to perform bitwise operations, e.g., taking the conjunction and disjunction of corresponding bits in a pair of registers, taking the negation of each bit in a register, or to compare two values in registers (for example, to see if one is less, or if they are equal).

Control flow operations, which may include an instruction to branch to another location in the program and execute instructions there, conditionally branch to another location if a certain condition holds, indirectly branch to another location, or call another block of code, while saving the location of the next instruction as a point to return to.

Coprocessor instructions, which may include an instruction to load/store data to and from a coprocessor, or exchanging with CPU registers, or perform coprocessor operations.

A processor of a computer of the present system may include “complex” instructions in their instruction set. A single “complex” instruction does something that may take many instructions on other computers. Such instructions are typified by instructions that take multiple steps, control multiple functional units, or otherwise appear on a larger scale than the bulk of simple instructions implemented by the given processor. Some examples of “complex” instructions include: saving many registers on the stack at once, moving large blocks of memory, complicated integer and floating-point arithmetic (sine, cosine, square root, etc.), SIMD instructions, a single instruction performing an operation on many values in parallel, performing an atomic test-and-set instruction or other read-modify-write atomic instruction, and instructions that perform ALU operations with an operand from memory rather than a register.

An instruction may be defined according to its parts. According to more traditional architectures, an instruction includes an opcode that specifies the operation to perform, such as add contents of memory to register—and zero or more operand specifiers, which may specify registers, memory locations, or literal data. The operand specifiers may have addressing modes determining their meaning or may be in fixed fields. In very long instruction word (VLIW) architectures, which include many microcode architectures, multiple simultaneous opcodes and operands are specified in a single instruction.

Some types of instruction sets do not have an opcode field (such as Transport Triggered Architectures (TTA) or the Forth virtual machine), only operand(s). Other unusual “0-operand” instruction sets lack any operand specifier fields, such as some stack machines including NOSC.

Conditional instructions often have a predicate field—several bits that encode the specific condition to cause the operation to be performed rather than not performed. For example, a conditional branch instruction will be executed, and the branch taken, if the condition is true, so that execution proceeds to a different part of the program, and not executed, and the branch not taken, if the condition is false, so that execution continues sequentially. Some instruction sets also have conditional moves, so that the move will be executed, and the data stored in the target location, if the condition is true, and not executed, and the target location not modified, if the condition is false Similarly, IBM z/Architecture has a conditional store. A few instruction sets include a predicate field in every instruction; this is called branch predication.

The instructions constituting a program are rarely specified using their internal, numeric form (machine code); they may be specified using an assembly language or, more typically, may be generated from programming languages by compilers.

Nothing in the foregoing should be interpreted to mean that any advantage of the present systems and methods is achievable by all embodiments of the present invention.

1. An appliance for assisting locomotion of a subject in need thereof, the appliance comprising:
paired ankle joint control means configured to return the ankle joint to a neutral position during locomotion.

2. The appliance of claim 1 wherein the neutral position is where the long axis of the lower leg is about 90 degrees to a plane described by the sole of the foot.

3. The appliance of claim 1 wherein the paired ankle joint control means returns the ankle joint to a neutral position by way of a biasing means.

4. The appliance of claim 3 wherein the biasing means is a torsion spring.

5. The appliance of claim 1 comprising paired electromechanical knee joint control means configured to move each knee joint of the subject through a range of motion required for locomotion,

6. The appliance of claim 5 wherein each of the paired electromechanical knee joint control means comprises biasing means configured to substantially counteract some or all of the gravitational force occasioned on the subject when moving from a seated position to a standing position, so as to extend the knee joint and allow the subject to assume a standing position.

7. The appliance of claim 6 wherein the biasing means is a leaf spring or a torsion spring.

8. The appliance of claim 6 wherein each of the paired electromechanical knee joint control means comprises at least one electric motor configured to effect the range of motion of the knee joint required for locomotion.

9. The appliance of claim 8 wherein each of the paired electromechanical knee joint control means comprises one or more of a gear, a drive, a pulley; each of which is in direct or indirect operable connection with the at least one electric motor; the one or more of a gear, a drive, a pulley being configured to effect the range of motion of the knee joint required for locomotion.

10. The appliance of claim 1 comprising paired electromechanical hip joint control means configured to move each hip joint through a range of motion required for locomotion,

11. The appliance of claim 10 wherein each of the paired electromechanical hip joint control means comprises at least one electric motor configured to effect the range of motion of the hip joint required for locomotion.

12. The appliance of claim 11 wherein each of the paired electromechanical hip joint control means comprises one or more of a gear, a drive, a pulley; each of which is in direct or indirect operable connection with the at least one electric motor; the one or more of a gear, a drive, a pulley being configured to effect the range of motion of the knee joint required for locomotion.

13. The appliance of claim 10 wherein each of the paired electromechanical hip joint control means is configured to rotate the femur of the subject forwards and/or backwards and/or outwards and/or inwards and/or effect axial rotation of the femur.

14. The appliance of claim 5 comprising electronic appliance control means in electrical or data communication with the paired electromechanical knee joint control means, the electronic appliance control means configured to coordinate the movement of each knee joint of the subject so as to cause or assist locomotion of the subject.

15. The appliance of claim 14 comprising knee joint sensing means configured to sense a degree of bending or straightening of the knee, the sensing means being in electrical or data communication with the electronic appliance control means.

16. The appliance of claim 10 comprising:
electronic appliance control means in electrical or data communication with the paired electromechanical hip joint control means, the electronic appliance control means configured to coordinate the movement of each hip joint of the subject so as to cause or assist locomotion of the subject; and
hip joint sensing means configured to sense a degree of movement and/or direction of movement and/or rotation of the femur of the subject relative to the pelvis of the subject, the sensing means being in electrical or data communication with the electronic appliance control means.

17. The appliance of claim 16, wherein the paired electromechanical hip joint control means is configured to operate in a coordinated manner via the electronic appliance control means so as to move the pelvis forwards and/or backwards and/or outwards and/or inwards and/or effect axial rotation of the pelvis.

18. The appliance of claim 15 wherein the electronic appliance control means accepts electrical signals and/or data from the knee joint sensing means, and is configured to process the electrical signals and/or data so as to provide output electrical signals and/or data, the output electrical signals and/or data being communicated to any of the one or more motors of each of the paired electromechanical knee joint control means so as to cause or assist for locomotion of the subject.

19. (canceled)

20. (canceled)

21. (canceled)

22. A method comprising:
providing a locomotion-capable subject,
providing paired knee joint sensing means configured to sense the degree of bending or straightening of a knee of the subject,
providing paired hip joint sensing means configured to sense the degree of movement and/or direction of movement and/or rotation of a femur relative to a pelvis of the subject,
attaching the knee joint sensing means to each knee joint and the hip joint sensing means to each hip joint of the subject,
allowing the locomotion-capable subject to move from a seated position to a standing position and/or walk and/or jog and/or run and/or move from a standing position to seated position,
recording over a period of time electrical signals and/or data output by the paired knee joint sensing means and the paired hip joint sensing means so as to identify a normal range of motion of each of the knee joints and the hip joints of the locomotion-capable subject during any of moving from a seated position to a standing position and/or walking and/or jogging and/or running and/or moving from a standing position to seated position, and storing the recorded electrical signals and/or data in a non-transitory computer-readable medium.

23. The method of claim 22, further comprising:
attaching paired ankle joint control means to the subject for returning the ankle joint to a neutral position during locomotion;
executing processor executable software stored on the computer-readable medium by a processor of a control device, which when executed by the processor accepts as input the stored electrical signals and/or data and configures the control device to control the paired ankle joint control means so as to effect a required range of motion in the paired ankle joint control means.
